(19) United States
(12) Patent Application Publication
(10) Pub. No.: US 2016/0367766 A1
(43) Pub. Date: Dec. 22, 2016

(54) INJECTION TRAINING AND COMPLIANCE DEVICE AND METHOD

(71) Applicants: Jeff BAKER, Orlando, FL (US); Craig BAKER, Lebanon, OH (US); Wai Yin Christopher CHUNG, Orlando, FL (US); Seth FREYTAG, Winter Springs, FL (US); Francis Michael SIEMER, Orlando, FL (US); Dinesh Venkata KOKA, Altamonte Springs, FL (US)

(72) Inventors: Jeff BAKER, Orlando, FL (US); Craig BAKER, Lebanon, OH (US); Wai Yin Christopher CHUNG, Orlando, FL (US); Seth FREYTAG, Winter Springs, FL (US); Francis Michael SIEMER, Orlando, FL (US); Dinesh Venkata KOKA, Altamonte Springs, FL (US)

(21) Appl. No.: 15/079,456

(22) Filed: Mar. 24, 2016

Related U.S. Application Data
(60) Provisional application No. 62/137,354, filed on Mar. 24, 2015.

(51) Int. Cl. A61M 5/42

(52) U.S. Cl. CPC A61M 5/427

(57) ABSTRACT

In an embodiment, a positioning apparatus for guiding an injection device relative to a target surface having a representative plane is provided. The apparatus includes a housing defining a channel configured to receive an injection device, wherein a first angle is formed between a longitudinal axis of the channel and the representative plane of the target surface so as to guide the injection device through the channel to the target surface at the first angle, and wherein the housing comprises a stabilizing surface configured to abut the target surface to stabilize an injection according to the first angle.

### BACKGROUND

The use of needles and instruments containing needles are widespread in the medical industry. Needle insertion into a patient is an essential component of many clinical procedures such as biopsies, injections, neurosurgery, and cancer treatment methods. Certain disease states require daily injections, such as diabetes mellitus. The success of these procedures depends on accurate placement and correct technique during insertion of the needle into a target region of a patient. Accuracy of an injection can be hampered by certain factors, including a fear of needles leading to failure to administer an injection, or improper administration of an injection. Lack of knowledge and/or experience in administering injections to oneself or to others may also lead to improper injection administration.

Increased use of biologic and large molecule drugs has driven the demand for prefilled syringes and other drug delivery systems. Prefilled syringes are combination products used in home and institutional settings by patients and health care providers (HCP). Failure to effectively use a prefilled syringe, for example, can result in errors that adversely affect the safe and full delivery of a prescribed dose. Training users of prefilled syringes and other needle-bearing drug delivery systems serves as an attractive strategy to mitigate errors and support the commercial objectives of syringe products and other medication administration products in the market.

Injectable medications are required for a number of varying illnesses and diseases. Many injectable medications require self-injection by a patient. Self-injection of a medicament using a device having a needle carries with it a certain stigma. Oftentimes users are weary of administering an injection for fear or anxiety related to handling an injection device, failing to deliver a complete dose of the medication, anticipated pain associated with injecting the subject with the needle, fear of accidentally sticking themselves with the needle during manipulation of the injection device, and difficulties in adequately grasping the dosing mechanism or injection device to inject the subject, among other concerns. An additional concern exists in instances in which users with little or no medical knowledge or experience are required to inject themselves or another subject using these devices. Performing a medical treatment or test using a device having or requiring a needle carries with it certain risks and often creates a level of anxiety for the user performing the treatment or test and/or for the subject receiving the treatment or test. Injections are often a source of fear and apprehension for injection providers as well as patients receiving injections. Studies suggest that as many as one-fifth to one-third of people with diabetes, for example, are hesitant or unwilling to give themselves insulin injections for reasons that include needle anxiety^{1,2,3} ¹ Zambanini, A., Newson, R. B., Maisey, M., Feher, M. D. (1999). Injection related anxiety in insulin-treated diabetes. Diabetes Research and Clinical Practice, 46, 239-246.² Klobassa, N., Moreland, P. (2012, Jan. 10). Psychological insulin resistance stems from fear. MayoClinic.com Living with Diabetes Blog, Message posted to http://www.mayoclinic.com/health/psychological-insulin-resistance/MY01165³ Nam, S., Chesla, C., Stotts, N. A., et al. (2010). Factors associated with psychological insulin resistance in individuals with Type 2 diabetes. Diabetes Care, 33, 1747-1749.)

Additional concerns arise where particular steps must be followed during an injection, or where the correct positioning of the injection device must be used to prevent harm to the patient and to administer an injection correctly. Un-trained injection providers may not appreciate the deleterious effects that an improperly administered injection may cause including, but not limited to trauma to bone tissue caused by contact with the needle, systemic poisoning as a result of incorrect placement of anesthetic injections and injection into the blood stream, for example, hematoma formation resulting from incorrect placement of the needle followed by aspiration during injections during anesthetic procedures, among other detrimental results of improper technique in positioning and placement of injection devices.

### SUMMARY

In an embodiment, a positioning apparatus for guiding an injection device relative to a target surface having a representative plane is provided. The apparatus includes a housing defining a channel configured to receive an injection device, wherein a first angle is formed between a longitudinal axis of the channel and the representative plane of the target surface so as to guide the injection device through the channel to the target surface at the first angle, and wherein the housing comprises a stabilizing surface configured to abut the target surface to stabilize an injection according to the first angle.

In a further embodiment, a positioning apparatus for guiding an injection device relative to a target surface is provided, wherein the apparatus includes a housing including a body having a generally conical shape, the body comprising a base portion, an apex portion, an outer surface and an inner surface, wherein an aperture is defined at the apex portion. The apparatus further includes a channel disposed adjacent to the inner surface, the channel terminating at the aperture in the apex portion, the channel configured to receive an injection device, wherein a predetermined angle is formed between a longitudinal axis of the channel and a longitudinal axis of the outer surface defined between the base portion and the apex portion, such that when the outer surface of the body is positioned against the target surface, the injection device contacts the target surface through the aperture at the predetermined angle.

In still a further embodiment, a positioning apparatus for guiding an injection device relative to a target surface is provided. The apparatus includes a housing including a first side having an upper edge portion and a lower edge portion, a second side having an upper edge portion and a lower edge portion, and a bottom surface having a first edge portion and a second edge portion. The first edge portion of the bottom surface associates with the lower edge portion of the first side, and the second edge portion of the bottom surface associates with the lower edge portion of the second side defining an opening there between. A visual indicator may be provided on the first and/or second side, wherein the visual indicator may include a marking on the housing, in a non-limiting embodiment, wherein the marking defines an angular placement of an injection device relative to a longitudinal axis of the bottom surface, such that when aligned with the marking, a first angle is formed between a longitudinal axis of the injection device and the longitudinal axis of the bottom surface of the housing.

In yet a further embodiment, a housing having a support surface for guiding an injection device is provided, wherein the housing is configured to facilitate control of the vector of the injection device relative to a target surface having a representative plane at a first angle relative to the representative plane. The housing includes a stabilizing surface configured to abut the target surface to stabilize an injection at the target surface according to the first angle.

In still a further embodiment, an injection device including a housing having a support surface for guiding an injection device to a target point on a target surface having a representative plane, wherein the housing is configured to facilitate control of the vector of the injection device relative to a target surface at a first angle, the housing comprising a stabilizing surface configured to abut the target surface to stabilize an injection at the target surface according to the first angle.

In another embodiment, a method for providing control of an injection device at a target point of a target surface along a first angle relative to the target surface, the target surface having a representative plane is provided. The method includes positioning a positioning apparatus comprising a housing having a guide and being configured to receive an injection device, wherein the guide forms a first angle between a longitudinal axis of the guide and the representative plane of the target surface so as to guide an injection device to the target surface at the first angle and wherein said housing includes a stabilizing surface to stabilize an injection at the target surface according to the first angle, such that the stabilizing surface of the housing abuts the target surface, and a distal portion of the guide is in alignment with the target point of the target surface, placing the injection device onto the housing in alignment with the guide at the first angle, and gliding the injection device to the target point.

### BRIEF DESCRIPTION OF THE DRAWINGS

A more particular description briefly stated above will be rendered by reference to specific embodiments thereof that are illustrated in the appended drawings. Understanding that these drawings depict only typical embodiments and are not therefore to be considered to be limiting of its scope, the embodiments will be described and explained with additional specificity and detail through the use of the accompanying drawings in which:

FIGS. 1A-1B include side views of an embodiment of a positioning apparatus for guiding an injection device relative to a target surface.

FIGS. 1C-1D are perspective views of the embodiment shown in FIGS. 1A-1B.

FIGS. 2A-2B include side views of another embodiment of a positioning apparatus for guiding an injection device relative to a target surface.

FIGS. 3A-3B include side views of a further embodiment of a positioning apparatus for guiding an injection device relative to a target surface.

FIG. 3C is a perspective view of the embodiment of the positioning apparatus shown in FIGS. 3A-3B.

FIGS. 4A-4D include side views of still a further embodiment of a positioning apparatus for guiding an injection device relative to a target surface.

FIGS. 5A-5B include a side view of another embodiment of a positioning apparatus for guiding an injection device relative to a target surface.

FIGS. 5C-5D are perspective views of the embodiment of the positioning apparatus shown in FIGS. 5A-5B.

FIGS. 6A-6B include side views of an embodiment of a positioning apparatus for guiding an injection device relative to a target surface having a visual indicator.

FIG. 7 includes a side view of an embodiment of a positioning apparatus comprising a sensor in wireless communication with an external device.

FIGS. 8A-8B include side views of an embodiment of a positioning apparatus associated with an attachment component.

FIGS. 9A-9C include side views of an embodiment of an adjustable positioning apparatus.

FIG. 10 includes a perspective view of an embodiment of a positioning apparatus having a platform extending from a stabilizing surface.

FIG. 11 provides an illustration of an example of a target surface having a target point, showing a non-limiting example of a representative plane on the target surface.

FIG. 12 provides a bottom view of a non-limiting embodiment of a positioning apparatus providing at least two points (a, b,) which contact the target surface.

FIG. 13A is a side view of a further embodiment of a positioning apparatus.

FIG. 13B is a front view of the embodiment shown in FIG. 13A.

FIG. 13C is a perspective view of the embodiment shown in FIGS. 13A-13B.

FIGS. 13D-13F show perspective, side and front views of the positioning apparatus shown in FIGS. 13A-13C in use.

FIGS. 14A-14C provide side, front and perspective views, respectively, of a further embodiment of a positioning apparatus.

FIGS. 14D-14F provide perspective, side and front views, respectively, of the embodiment of the positioning apparatus in FIGS. 14A-14C in use.

FIG. 15A is a top view of a further embodiment of a positioning apparatus.

FIG. 15B is a perspective view of the positioning apparatus embodiment shown in FIG. 15A.

FIG. 15C is a side view of the positioning apparatus embodiment shown in FIG. 15A.

FIG. 15D is a front view of the positioning apparatus embodiment shown in FIG. 15A.

FIG. 15E is a bottom view of the positioning apparatus embodiment shown in FIG. 15A.

### DETAILED DESCRIPTION

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains.

It is to be noted that the terms “first,” “second,” and the like as used herein do not denote any order, quantity, or importance, but rather are used to distinguish one element from another. The terms “a” and “an” do not denote a limitation of quantity, but rather denote the presence of at least one of the referenced item. The modifier “about” used in connection with a quantity is inclusive of the stated value and has the meaning dictated by the context (e.g., includes the degree of error associated with measurement of the particular quanity). It is to be noted that all ranges disclosed within this specification are inclusive and are independently combinable.

The terminology used herein is for the purpose of describing particular embodiments only and is not intended to be limiting. As used herein, the singular forms “a,” “an,” and “the” are intended to include the plural forms as well, unless the context clearly indicates otherwise these terms do not denote a limitation of quantity, but rather denote the presence of at least one of the referenced item. Furthermore, to the extent that the terms “including,” “includes,” “having,” “has,” “with,” or variants thereof are used in either the detailed description and/or the claims, such terms are intended to be inclusive in a manner similar to the term “comprising.” Moreover, unless specifically stated, any use of the terms first, second, etc., does not denote any order, quantity or importance, but rather the terms first, second, etc., are used to distinguish one element from another.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective measurements. Moreover, all ranges disclosed herein are to be understood to encompass any and all sub-ranges subsumed therein. As a non-limiting example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 7.

### DEFINITIONS

The term “attachment component” as provided herein includes, but is not limited to an adhesive, a structural attachment means such as a gripping device that may be associated with or wrapped around a portion of a user to position the positioning apparatus, housing, and/or injection device against a target surface of the user. In one non-limiting example, the attachment component may include a bracelet-type structural device, which may be removably affixed to a user by placing around a limb of a user during an injection event. In another non-limiting embodiment, the attachment component may include an adhesive material associated with a portion of the stabilizing surface, configured to attach the positioning apparatus to a target surface of a user.

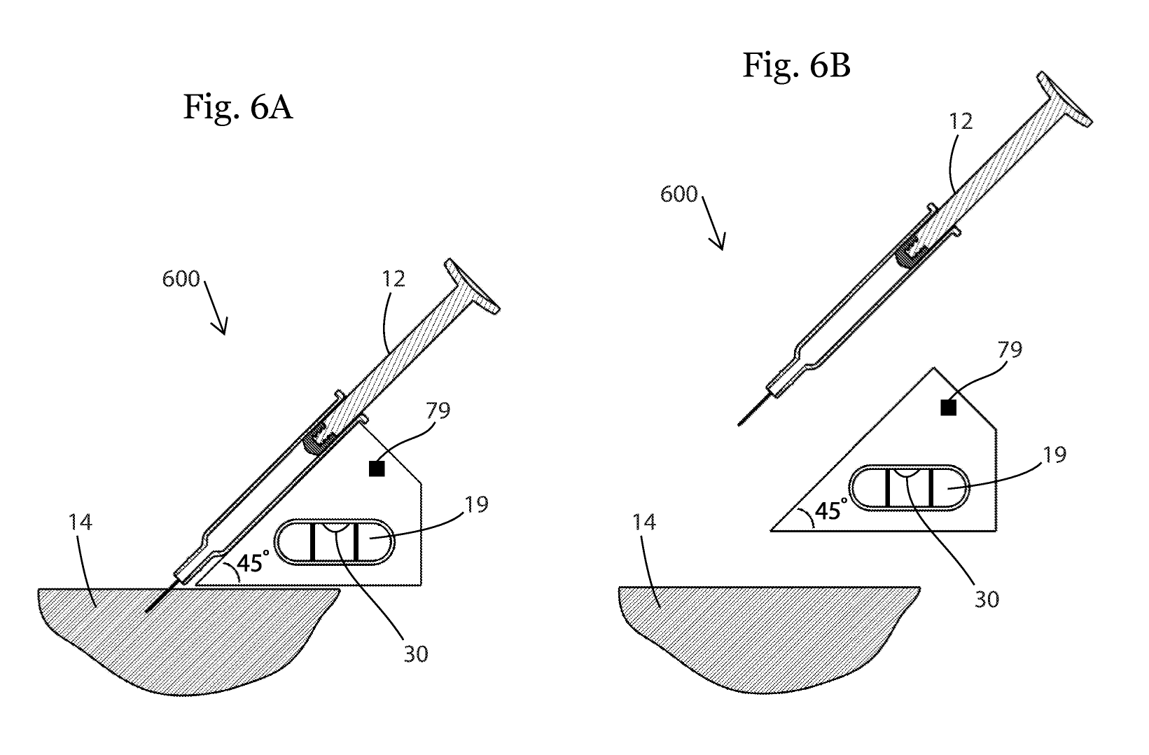

The term “visual indicator” as provided herein includes, but is not limited to, a marking such as an etching or a sticker or other type of marking known to those skilled in the art on a portion of the housing, or a light indicator to guide a positioning of an injection device relative to the housing, in one non-limiting example. In another, non-limiting embodiment (as shown in FIG. 6A-B), the visual indicator may include a level-component or other such device for guiding an alignment of the injection device relative to the target surface. The level-component may be disposed on a surface of the housing to guide a user in positioning the apparatus relative to the target surface at the first angle, wherein at the first angle, a bubble may be disposed in the center of the level-component.

The term “about” as used herein includes a 5-10% or 5-10 degree variance.

The term “representative plane” as used herein in reference to the target surface includes a tangential plane to a normal on the target surface. For illustration purposes, in one example, a representative plane is a tangential plane to the normal of the insertion point on the target surface. that is an average taken from multiple varying levels of the representative surface, for example, quite often a patient tissue target surface does not lie in one plane. Consequently, the representative plane includes an average plane of the target surface.

The term “injection device” as used herein includes a needle-containing or a non-needle containing device. Injection device includes medicament delivery devices as well as training devices, and any combination of the two.

The term “condition” as used herein includes but is not limited to a user input, a status of the housing, positioning apparatus and/or injection device, anything that is sensed by the housing, positioning apparatus and/or injection device, correct or incorrect stepwise activities, usage of the housing, positioning apparatus and/or injection device, usage of the housing, positioning apparatus and/or injection device over time, among other conditions. The term “condition” is configured o include an “error condition”, which includes but is not limited to a condition pertaining to a mistake by the user in using the housing, positioning apparatus and/or injection device, whether the mistake is incorrect positioning or contact between the housing, positioning apparatus and/or injection device and the user, or whether the mistake is an out of order step, a step that exceeds or fails to meet predetermined time value (such as an undue pause during or between steps, or insufficient time for conducting a step or transition between steps). Error conditions may also include errors of the housing, positioning apparatus and/or injection device, including low or lack of power or failure to operate as intended.

The term “associated” or “association”, as used herein, includes but is not limited to direct and indirect attachment, adjacent to, in contact with, partially or fully attached to, and/or in close proximity therewith. The term “value” as used herein, may refer to a specific value or a range of values.

A “predetermined value” as used herein, for example, includes but is not limited to a value or range of values relating to an event involving use or operation of the housing, positioning apparatus and/or injection device. These may include, but are not limited to thresholds, ceilings, baselines or range values that are desired or undesired for a particular event. Examples of predetermined values include, but are not limited to, a predetermined orientation value, predetermined time value, or a predetermined contact value, in a particular example, predetermined value may refer to a predetermined angle value as described herein, in addition to other predetermined values described herein refers to a value that is used as a reference value in relation to a value, signal, or indication that is detected by, for example, a sensor of the housing, positioning apparatus and/or injection device. Predetermined value may include an optimal value, or a sub-optimal value, or any value there between.

In one non-limiting example, a predetermined value may include a 90 degree angle between the injection device, positioning apparatus and/or housing, and a target region for the injection, an additional predetermined value may include a 10 degree angle between the device and a target region for the housing, positioning apparatus and/or injection device, for example. At either predetermined value, or at any value there between, a signal output component may be initiated. The signal output component may therefore be an error message or a congratulatory message, for example.

The term “use” as used herein includes, but is not limited to, operation of the housing, positioning apparatus and/or injection device.

As used herein, the terms “subject”, “user” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human.

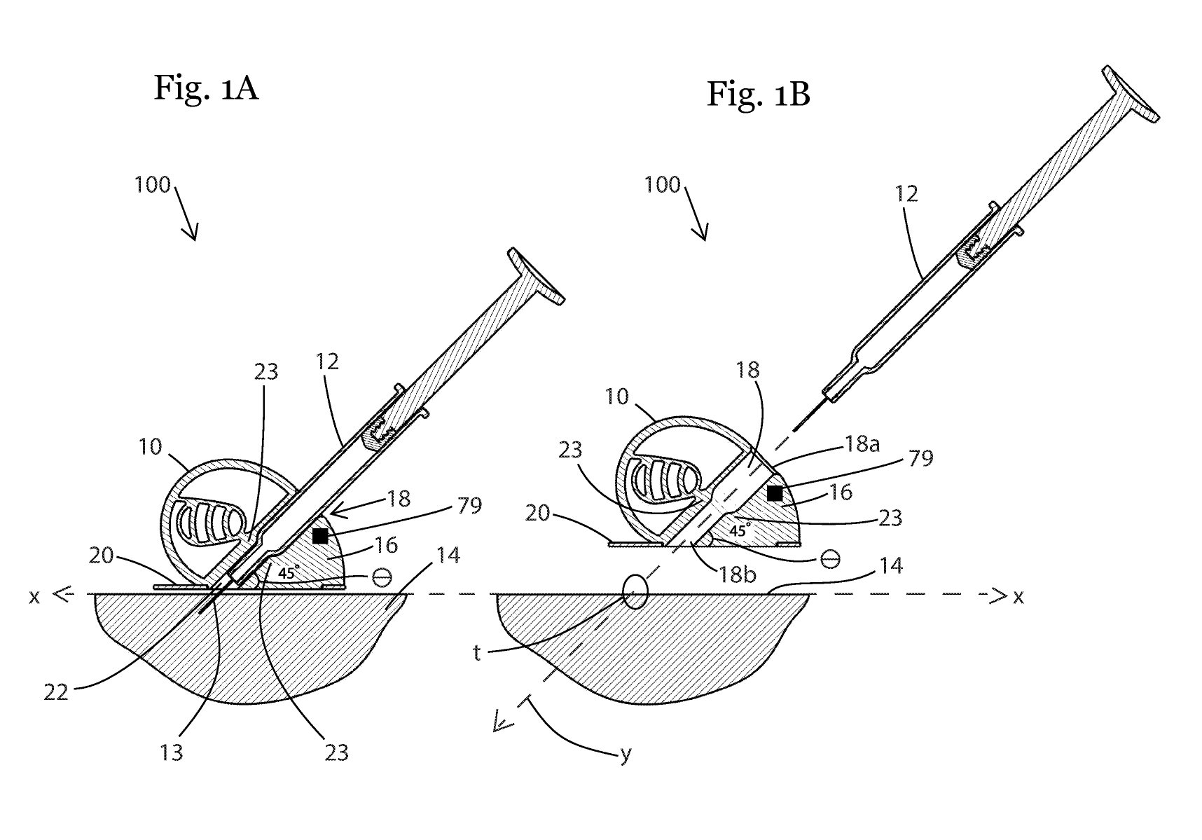

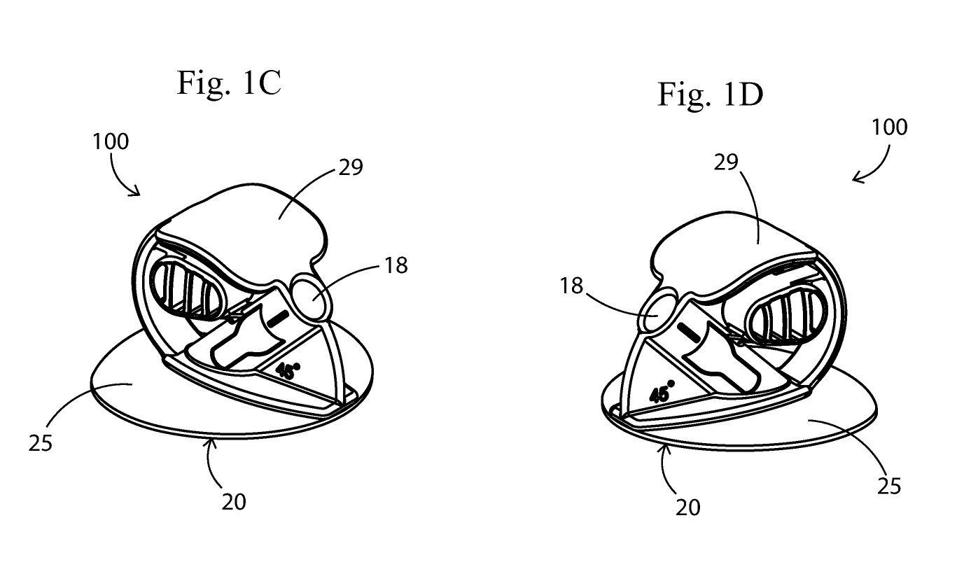

Turning to the Figures, FIGS. 1A-1B include side views of an embodiment 100 of a positioning apparatus 10 for guiding an injection device 12 relative to a target surface 14 having a representative plane x, the apparatus 10 includes a housing 16 defining a channel 18 configured to receive an injection device 12, wherein a first angle θ is formed between a longitudinal axis of the channel y and the representative plane x of the target surface 14 so as to guide the injection device 12 through the channel 18 to the target surface 14 at the first angle θ, and wherein the housing 16 comprises a stabilizing surface 20 configured to abut the target surface 14 to stabilize an injection according to the first angle θ. In a non-limiting embodiment, the first angle θ may include about a 90 degree angle. In another non-limiting embodiment, as shown in FIG. 1A-B, the first angle may include about a 45 degree angle, such the positioning apparatus 10 may facilitate an injection at the 45 degree angle relative to the target surface 14. FIG. 1A provides a side view in which the injection device 12 is inserted into the channel 18 of the housing 16 of the positioning apparatus 10, such that a needle 13 of the injection device 12 traverses the target surface 14 at a 45 degree angle. In one embodiment, as shown in FIG. 1A, the positioning apparatus 10 may further include an aperture 22 formed in the stabilizing surface 20 of the housing 16 adjacent to the target surface 14, wherein when the injection device 12 is received within the channel 18, a portion of a distal end of the injection device 12 aligns with the aperture 22 in the housing 16, such that the needle 13 of the injection device 12 can exit the housing 16, traversing the housing 10 through the aperture 22 and entering the target tissue at the first angle θ.

FIG. 1B provides a view in which the injection device 12 has not been placed within the apparatus 10, and the apparatus 10 is not against the target surface 14. In further non-limiting embodiments, the first angle may include an angle between about 10-15 degrees, an angle less than 90 degrees, an angle between about 40 and 50 degrees, or an angle between 30 degrees and 90 degrees, for example. The stabilizing surface 20 reduces pivoting of the longitudinal axis y of the channel 18 relative to the target surface 14. The channel 18 includes a proximal end 18a, a distal end 18b, and the stabilizing surface 20 is configured to contact the target surface 14 at at least two points (a, b), at least 1-5 cm from a center point of a distal opening of the channel and in at least two non-parallel linear axes, in one non-limiting embodiment (see FIG. 12). In a further non-limiting embodiment, the linear axes form a stabilization angle θ there between that is at least 20 degrees (see FIG. 12). In a further embodiment, the positioning apparatus 10 is provided wherein the stabilizing surface 20 contacts the target surface 14 at at least three non-linear points, a, b, c, at least 1-5 cm from a center point of a distal opening.

In a further embodiment, the housing 16 may include one or more protrusions 23 adjacent to and/or extending into the channel 18. The one or more protrusions 23 are configured to retain the injection device 12 within the housing 16 when the injection device 12 is received within the channel 18.

In one non-limiting embodiment, the injection device 12 may include an injection training device. In another non-limiting embodiment, the injection device 12 may include a medicament delivery device, wherein the channel 18 is configured to guide the medicament delivery device to deliver an injection at a target point t at the target surface 14, such that a longitudinal axis of the medicament delivery device y is determined by the longitudinal axis of the channel 18, and the medicament delivery device contacts the target surface 14 at the first angle θ.

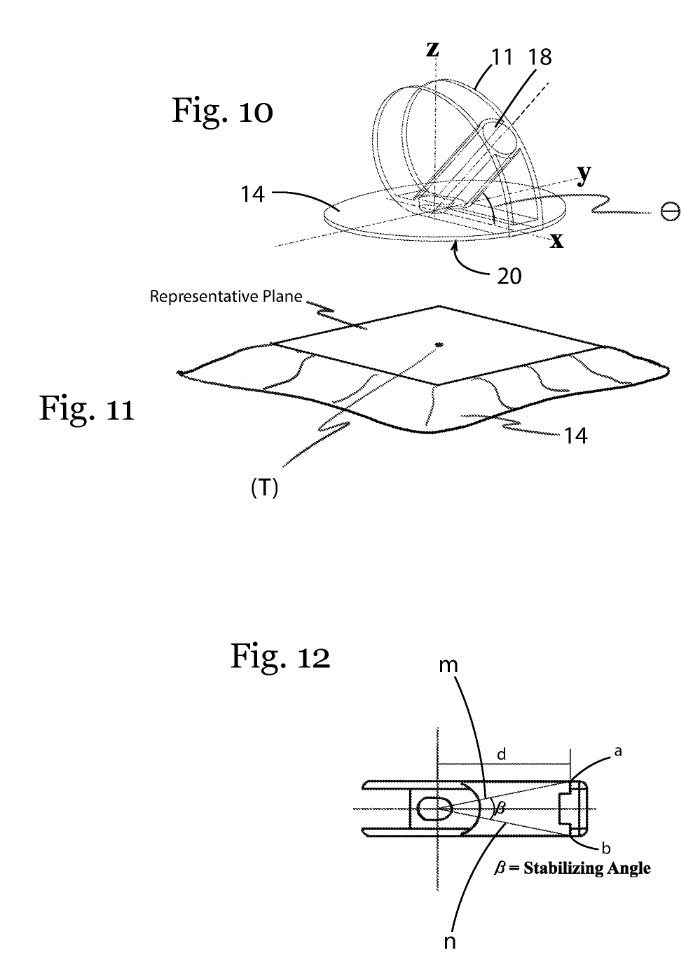

In a further embodiment (as shown in FIG. 10), the positioning apparatus 11 may include a platform 25 extending from the stabilizing surface 20, the platform 25 may include a longitudinal axis transverse to the longitudinal axis of the channel 18, and parallel to the longitudinal axis x of the target surface 14, wherein the platform 25 is configured to abut the target surface 14.

In still a further embodiment, the platform 25 may included a first portion 25a and a second portion 25b, said first and second portions 25a, 25b, are disposed on either side of the housing 16, and the platform 25 comprises a flexible material such that a user can grip the first and second portions of the platform to receive at least a portion of the target surface 14 between the first and second portions 25a, 25b of the platform 25 to displace the portion of the target surface 14 to receive the injection device 12. The flexible material may include, but is not limited to a rubber, a silicone, or any other deformable material known to those skilled in the art to which the invention pertains.

FIGS. 1C and 1D are perspective views of the positioning apparatus 100 showing the stabilizing surface 20 with an optional platform 25 extending outward from the stabilizing surface. The platform 25 may provide additional support to a user of the apparatus and may extend the stabilizing surface to further stabilize the apparatus during use. As described above, the platform 25 is configured to abut the target surface when the apparatus is in use. Furthermore, as described in some embodiments herein, the platform may optionally be made of a flexible material to allow a user to grip the area around and/or including the target area to provide an injection into the target area during use of the apparatus. The embodiment 100 may optionally include a safety guard 29 as shown in FIGS. 1C-1D, wherein the safety guard 29 may be provided to protect a user from needle prick during use of the apparatus, in one non-limiting embodiment. The safety guard 29 may also serve to visually guide the user to insert a portion of the injection device 12 (not shown in FIGS. 1C-1D), i.e., an injection member, into the channel 18 in a non-limiting embodiment.

Various safety features may be provided herein in regard to the positioning apparatus. One such safety feature includes the safety guard 29, wherein the portion of the user's hand that manipulates the positioning apparatus is protected from needle sticks from an injection device via the safety guard 29. At any point where a needle may come in contact with a user outside of the injection site, protection of the user is considered. Embodiments of the positioning apparatus described throughout this description also provide structural features for guiding an injection device via, in non-limiting examples, a visual guide, a channel within which the injection device may be received, or in another non-limiting example, walls of a positioning apparatus that prevent lateral movement and provide a lateral guide of the injection device during use, reduce or prevent risk of needle sticks during use of the apparatus with a needle-containing injection device.

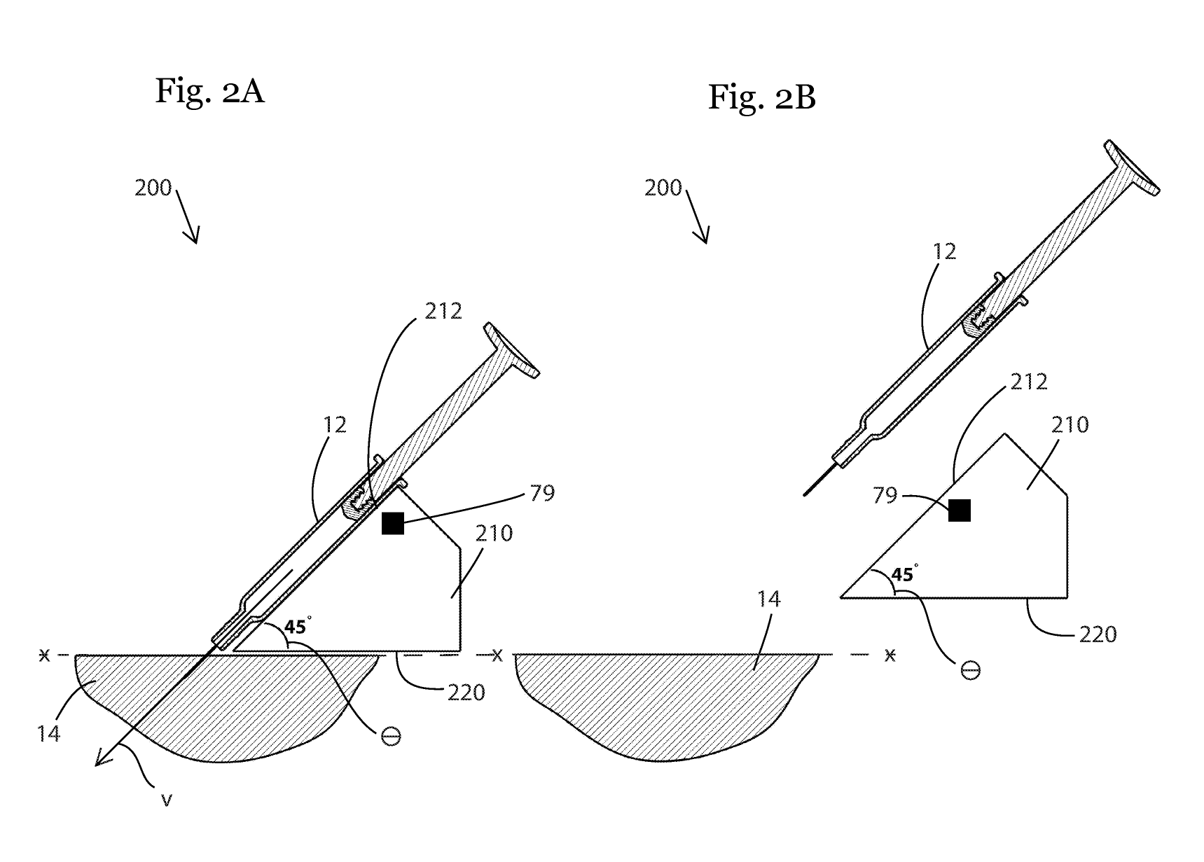

FIGS. 2A-2B include side views of an embodiment 200 of a housing 210 for guiding an injection device 12 relative to a target surface 14. The embodiment 200 includes a housing 210 having a support surface 212 for supporting and guiding the injection device 12, wherein the housing 210 is configured to facilitate control of the vector v of the injection device 12 relative to a target surface 14 having a representative plane x at a first angle θ relative to the representative plane x, wherein said housing comprises a stabilizing surface 220 configured to abut the target surface 14 to stabilize an injection at the target surface 14 according to the first angle θ.

In one embodiment, the vector v of the injection device 12 is less than a 90 degree angle relative to the representative plane x of the target surface 14. In a further embodiment, the vector v of the injection device 12 is between a 30 degree and a 90 degree angle relative to the representative plane x of the target surface 14. In another embodiment, the vector v of the injection device 12 is at about a 45 degree angle relative to the representative plane x of the target surface 14. In still another embodiment, the vector v includes about a 90 degree angle relative to the representative plane x of the target surface 14. In a further embodiment, the housing 210 may further include a platform portion 25 configured to abut a target surface 14. In still a further embodiment, the platform portion 25 may include a flexible material, the platform portion 25 having a first portion 25a and a second portion 25b, such that the platform 25 may be deformed around a portion of the target surface 14 received between the first portion 25a and the second portion 25b of the platform portion 25. The platform portion 25 may therefore be used to grip a layer of the target tissue between the first portion 25a and the second portion 25b of the platform 25 for receiving an injection in the target tissue.

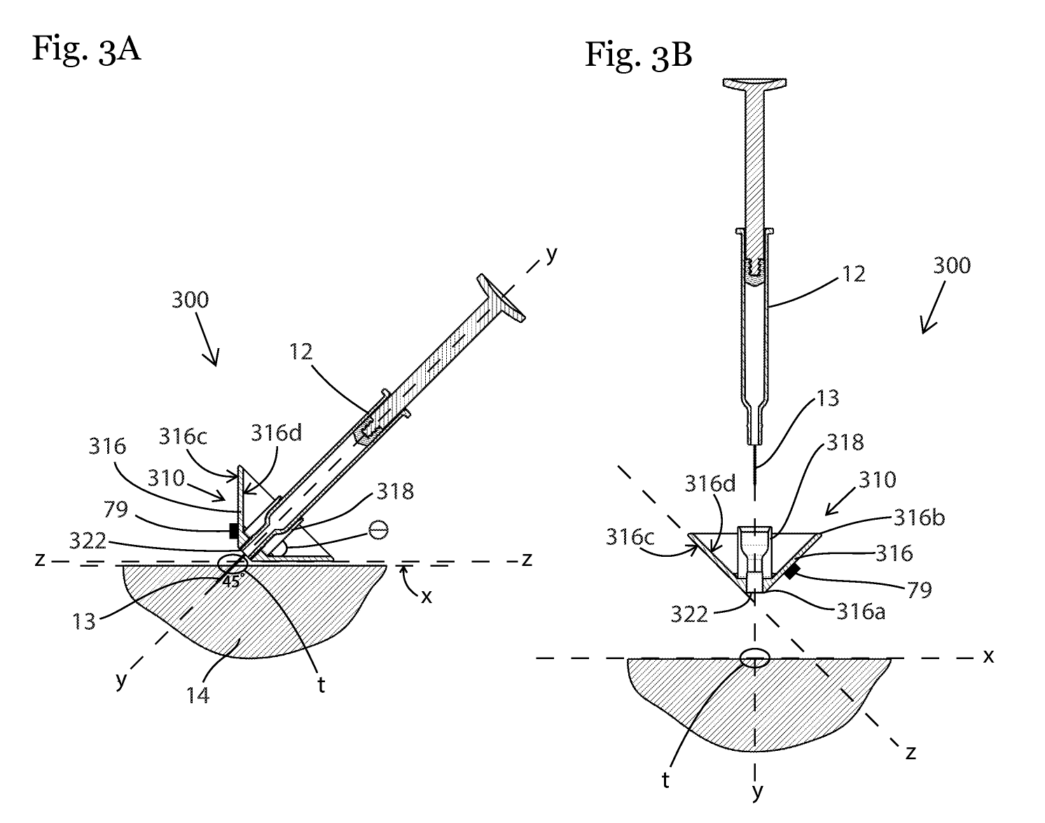

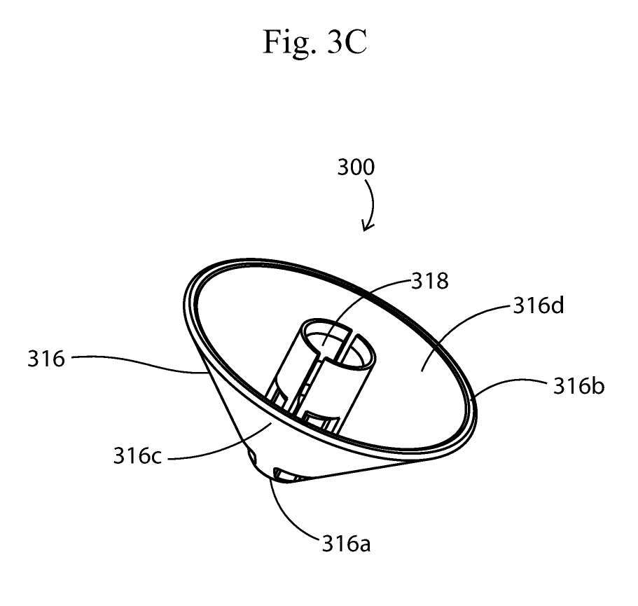

FIGS. 3A-3B include side views of a further embodiment 300 of a positioning apparatus 310 for guiding an injection device 12 relative to a target surface 14. The apparatus 310 includes a housing having a body 316 having a generally conical shape, the body 316 comprising a base portion 316b, an apex portion 316a, an outer surface 316c and an inner surface 316d, wherein an aperture 322 is defined at the apex portion 316a. A channel 318 disposed adjacent to the inner surface 316d, the channel 318 terminating at the aperture 322 in the apex portion 316a, the channel 318 is configured to receive an injection device 12, wherein a predetermined angle θ is formed between a longitudinal axis of the channel y and a longitudinal axis z of the outer surface 316c defined between the base portion 316b and the apex portion 316a of the body 316, such that when the outer surface 316c of the body 316 is positioned against the target surface 314 as shown in FIG. 3A, and an injection device 12 is placed in the channel 318, the injection device 12 contacts the target surface 14 through the aperture 322 at the predetermined angle θ. The predetermined angle θ in the non-limiting embodiment of FIG. 3A is 45 degrees.

When the body 316 of the positioning apparatus 310 is placed against the target surface 14, such that the outer surface 316c abuts the target surface 14, the longitudinal axis z of the outer surface 316c is generally aligned with the longitudinal axis of the representative plane x of the target surface 14 in the non-limiting embodiment of FIG. 3A. Consequently, the predetermined angle θ is formed between the longitudinal axis of the channel y and both the representative plane x of the target surface 14 and the longitudinal axis of the outer surface z in FIG. 3A. In one embodiment, the predetermined angle θ includes an angle less than 90 degrees. In another embodiment, the predetermined angle θ includes an angle between about 30 degrees and 90 degrees. In yet another embodiment, the predetermined angle θ is about 45 degrees as shown, for example in FIG. 3A.

The aperture 322 may be configured such that when the injection device 12 comprises a needle 13, the needle 13 may pass through the aperture 322 to a target point t on the target surface 14. The predetermined angle θ may include about a 90 degree angle, about a 45 degree angle, or about a 10-15 degree angle in non-limiting embodiments. In some examples, a 45 degree angle is used for subcutaneous injections. In other examples, a 45 degree angle may be used for intramuscular injections. In some instances a 90 degree angle is used during an injection with an auto-injector device, for example. FIG. 3B shows an embodiment in which the predetermined angle θ is about 90 degrees. Therefore, for example, in FIG. 3B, the predetermined angle θ is formed between the longitudinal axis of the channel y and the representative plane x of the target surface 14. In this non-limiting embodiment, the positioning apparatus 310 may be positioned against a target surface 14 at the target point such that a least a portion of its apex portion 316a contacts a portion of the target surface 14. In other non-limiting embodiments, for intra-dermal injections, for example, a 10-15 degree angle between the longitudinal axis of the injection device and the target surface is often preferred.

FIG. 3C is a perspective view of the embodiment 300 of the positioning apparatus shown in FIGS. 3A-3B. The embodiment 300 shown in FIG. 3C provides the body 316, having an apex 316a, a base 316b, an inner surface 316d and an outer surface 316c. The embodiment 300 also includes the channel 318 shown in FIG. 3C. When placed such that the base 316b is in contact with a target area of a user, as described above, an injection device 12 (not shown in FIG. 3C) can be received within the channel 318 to provide an injection or a simulated injection to a user at the target area, in one non-limiting embodiment. A predetermined angle θ formed between the injection device 12 and the surface of the user at the target area via the positioning apparatus 300 may include about a 45 degree angle, or about a 10-15 degree angle in non-limiting embodiments, depending on the size of the body 316, and the structural characteristics of the body 316, in a non-limiting example. The embodiment 300 may be placed, alternatively, such that the apex 316a abuts the target area t of the user (in the position shown in FIG. 3B when the apparatus is against the target point, t), and in this embodiment, the predetermined angle formed between the injection device 12 (not shown in FIG. 3C) when received within the channel 318, may include about a 90 degree angle between the injection device 12 and the target surface of the user (i.e., between the x and y axes as shown in FIG. 3B).

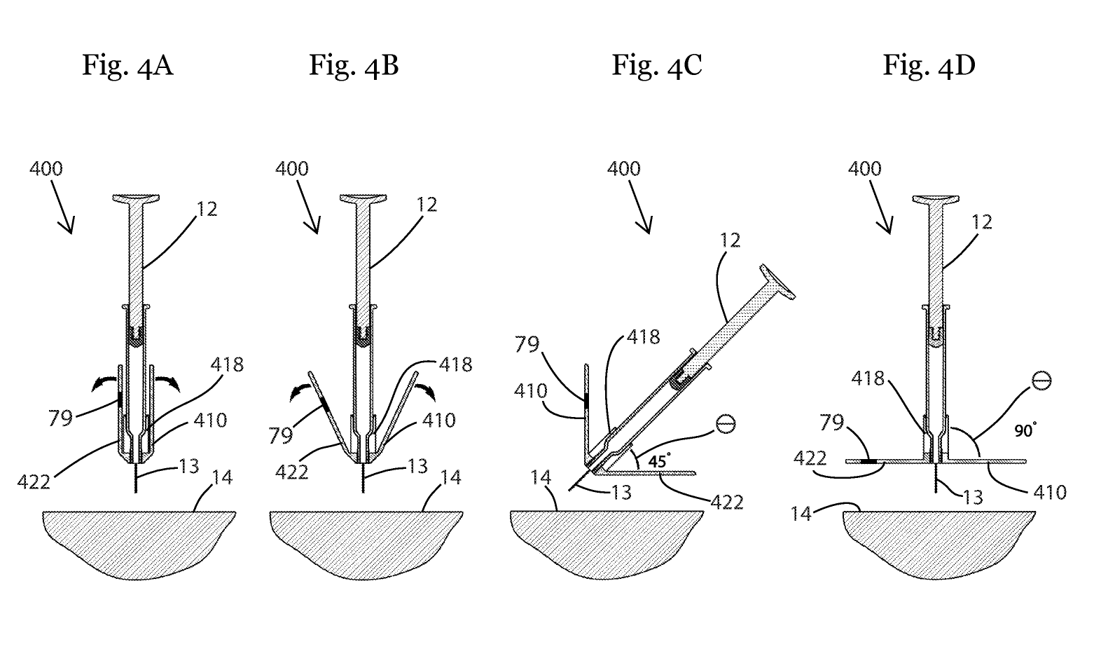

FIGS. 4A-4D include side views of still a further embodiment 400 of a positioning apparatus 410 for guiding an injection device 12 relative to a target surface 14. The positioning apparatus 410 includes a housing having an adjustable stabilizing surface 422, in one embodiment. The stabilizing surface 422 may include one or more components, which may be movable relative to a channel 418 of the positioning apparatus 410, to adjust a first angle θ formed between the channel 418 and the stabilizing surface 422. At least a portion of the stabilizing surface 412 is configured to abut a target surface 14 of a target tissue of a user, and the channel 418 is configured to receive an injection device 12 to guide the injection device 12 at the first angle θ to a target point t at the target surface 14 during an injection. The stabilizing surface 412 may be positioned such that the first angle θ includes a 45 degree angle as shown in FIG. 4C, in one non-limiting embodiment, or a 90 degree angle in another non-limiting embodiment as shown in FIG. 4D. Before and after use of the positioning apparatus 410, the stabilizing surface 422 may be positioned adjacent to the channel 418 as shown in FIG. 4A for storage and/or travel, for example. In one non-limiting embodiment, the positioning apparatus 410 may be affixed or attached to the injection device 12, or a component thereof.

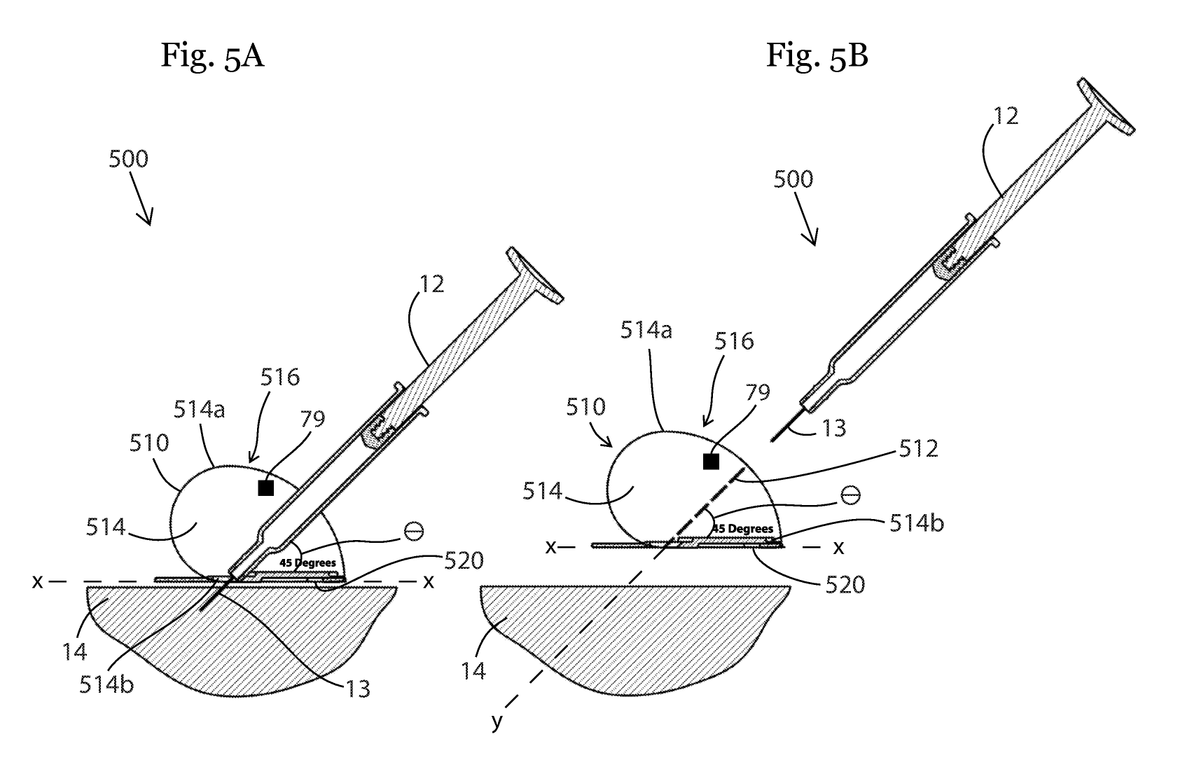

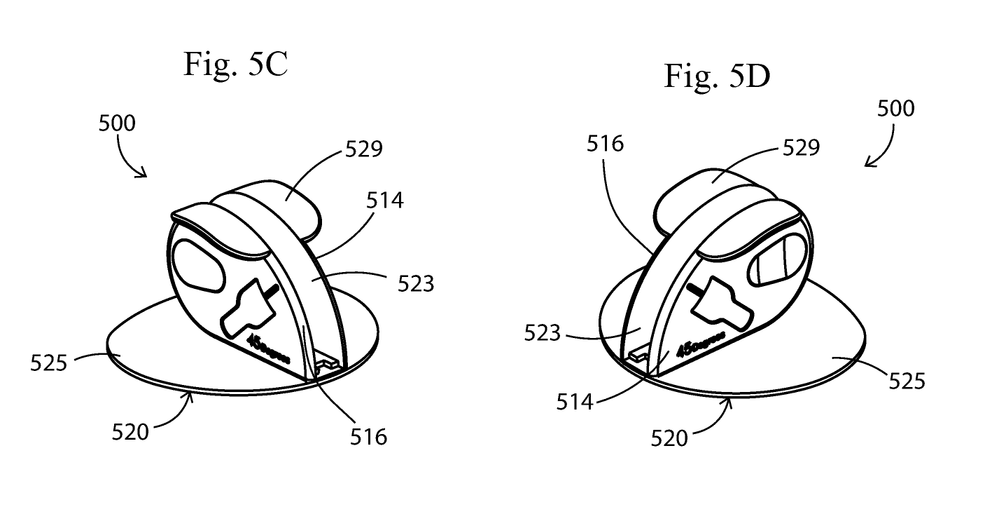

FIGS. 5A-5B include side views of another embodiment 500 of a positioning apparatus 510 for guiding an injection device 12 relative to a target surface 14, wherein the apparatus 510 includes a housing having a first side 514 having an upper edge portion 514a and a lower edge portion 514b, a second side 516 (not shown in FIG) having an upper edge portion 516a and a lower edge portion 516b, and a stabilizing surface 520 having a first edge portion 520a and a second edge portion 520b, wherein said first edge portion 520a of the stabilizing surface 520 associates with the lower edge portion 514b of the first side, and the second edge portion 520b of the stabilizing surface 520 associates with the lower edge portion 516b of the second side defining an opening 523 there between, and a visual indicator 512 on the first and/or second side 514, 516, wherein the visual indicator 512 may include a marking on the housing. The marking defines an angular placement of an injection device 12 relative to a longitudinal axis x of the stabilizing surface 520, such that when aligned with the marking, a first angle θ is formed between a longitudinal axis of the injection device y and the longitudinal axis of the stabilizing surface x.

In an embodiment, the stabilizing surface 520 is configured for placement on a patient against the target surface 14 for an injection. Placement of the injection device 12 in alignment with the visual indicator 512 guides the injection device 12 through the housing to the target surface 14 at the first angle θ. In a further embodiment, the stabilizing surface may include one or more apertures 522 for receiving an injection member 13 (i.e., a needle) of an injection device 12. In an embodiment, at least one of the one or more apertures 522 is disposed in the stabilizing surface 520 adjacent to the longitudinal axis of the injection device y when aligned with the visual indicator 512, the aperture 522 is configured to be associated with the target surface 14 and configured to receive the injection member 13 of the injection device 12. The injection device 12 may include an injection training device and/or an injection medicament delivery device. In a further embodiment, the stabilizing surface 520 may include a platform 525 configured to be positioned against a target surface 14 of a user. In a further embodiment, the platform may include a flexible material wherein a user can grip a portion of the target surface 14 between the flexible material of the platform 525 to raise the target surface 14 to receive the injection member 13. In one embodiment, the first angle may include about a 90 degree angle. In another embodiment, the first angle may include about a 45 degree angle. In still another embodiment, the first angle may include between about a 10-15 degree angle.

FIGS. 5C-5D include perspective views of the embodiment of the positioning apparatus 500 shown in FIGS. 5A-5B. The embodiment 500 includes the first side 514 and second side 516, the stabilizing surface 520 for placement against a user and an opening 523 between the first and second sides 514, 516 respectively, for receiving at least a portion of an injection device 512 (not shown). An optional safety guard 529 is shown in FIGS. 5C-5D, wherein the safety guard 529 protects a user from a potential needle stick when using the positioning apparatus with an injection device 12 having a needle, in an embodiment. The safety guard 529, also allows a user to grip onto the apparatus to position it against the target surface. Further, an optional platform 525 is shown extending from the stabilizing surface 520 of the embodiment 500. As described in reference to other embodiments, this platform 525 may further provide stabilization of the apparatus on the target surface of the user during use of the apparatus. Furthermore, the platform 525 may include a flexible material, such that it may be used by the user of the apparatus to grip a portion of the surface of the user surrounding the target area during use of the apparatus. In one non-limiting example, the platform 525 may be used to grip skin of the user surrounding or including the target area during an injection to better position a needle or injection member of the injection device 12 (not shown in FIGS. 5C-5D) into the target area, and to provide a more efficient injection into the target layer of the user (whether subcutaneous, intradermal, or other layer of the target area) as needed for effective deposition of the medicament contained within the injection device 12 for optimal therapy.

FIGS. 6A-6B include side views of one non-limiting embodiment of a visual indicator. The visual indicator may include a level-component 19 as shown in FIGS. 6A-B. The level component 19 may be provided to guide an injection device 12 to a target surface 14 at a predetermined angle θ. The level component 19 may be such that when an air bubble 30 is disposed in the center portion of the level-component 19, the correct predetermined angle θ between the injection device 12 and the target surface has been reached.

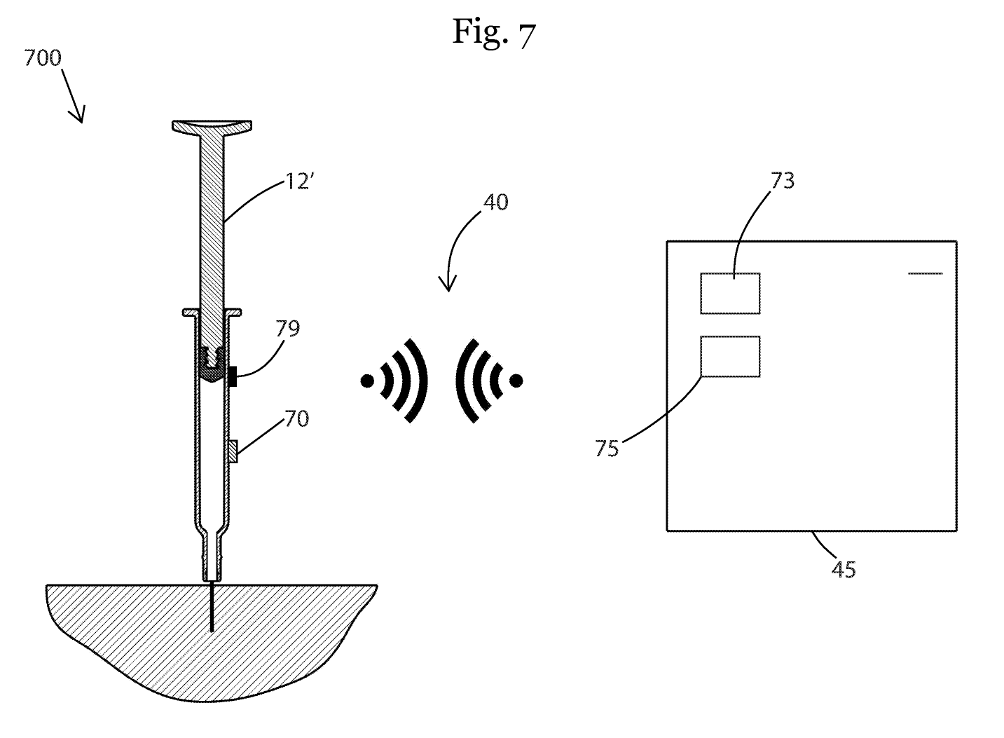

FIG. 7 includes a side view of a sensor embodiment of a positioning apparatus in wireless communication 40 with an external device 45. The external device 45 is an optional feature in this embodiment. The sensor embodiment 700 may include an electronic version of the positioning apparatus, having a position sensor 70, which may be embodied as an accelerometer or a tilt/angle sensor in non-limiting embodiments. There may further be an electronic chip on the injection device 12′, and communications with an external device 45 as shown in FIG. 7, or with the user directly by way of a feedback feature using visual, audio or other cues stimulating other senses to provide information to a user and communicate therewith. For example, based on information obtained by the sensor, the positioning apparatus may communicate to the user whether the angle at which an injection device is being positioned is correct or incorrect. This can be accomplished by any methods known in the art, including, for example, by providing a visual feedback by the user regarding the angle by way of either the external device 45 and/or an onboard feedback features via a signal output component 73, such as a light indicator or a speaker on the injection device, in non-limiting examples. Consequently, the positioning apparatus can detect correct or incorrect positioning and/or angle of the injection device and provide feedback to a user based on the detection, or communicate with the external device 45 via component 75 the information obtained, by sending a signal to the external device 45 from the injection device 12′ in one non-limiting embodiment.

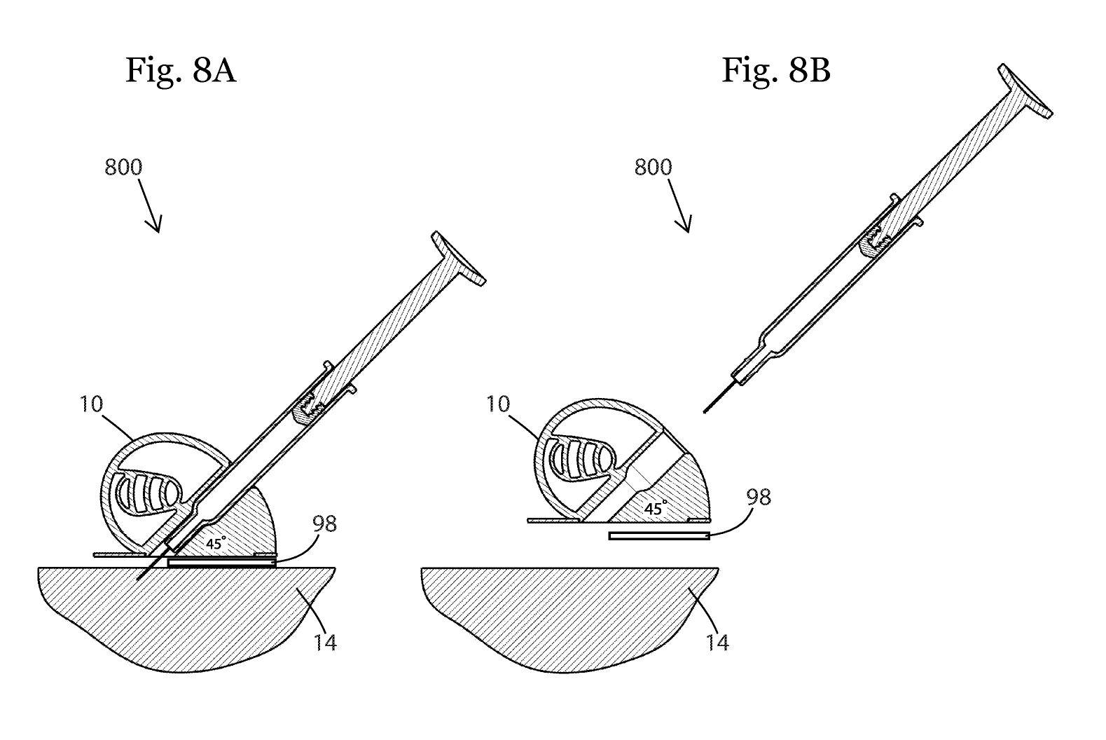

FIG. 8A-B shows side views of a non-limiting embodiment of a positioning apparatus including a housing 10 and an attachment component 98. The attachment component 98 is shown, in the non-limiting embodiment presented, as associated with a lower portion of the housing 10. The attachment component 98 can be adhered onto the target surface 14 of a user during use of the positioning apparatus, for example.

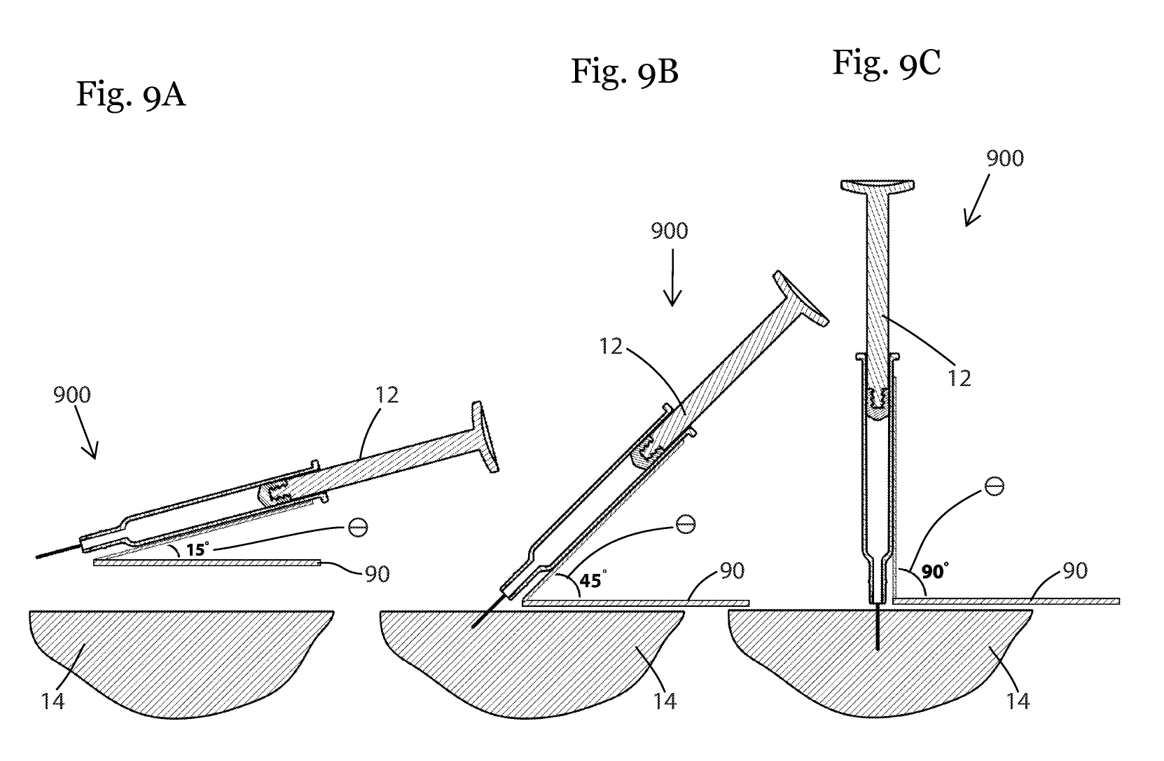

FIG. 9A-C includes side views of a non-limiting adjustable positioning apparatus embodiment 900, having a housing 90 which can be associated with a target surface 14 and an injection device 12 can be guided thereon for an injection at the target surface 14. The housing 90 may include a predetermined angle θ which may be adjustable as shown in FIGS. 9A-C. The angles shown are shown for example, only, and are not intended to be limiting of the embodiment provided herein.

FIG. 10 provides a non-limiting embodiment of a positioning apparatus 11 having a channel 18, a stabilizing surface 20, a predetermined angle θ, and a platform portion 25 extending from the stabilizing surface 20, and at least a portion of the platform portion 25 being configured to abut or associate with at least a portion of the target surface 14.

FIG. 11 provides an illustration of an example of a target surface 14 having a target point (T), showing a non-limiting example of a representative plane on the target surface 14.

FIG. 12 provides a bottom view of a non-limiting embodiment of a positioning apparatus providing at least two points (a, b,) which are configured to contact the target surface 14 at at least two points (a, b,), at a distance (d) of least 1-5 cm from a center point of a distal opening of the channel 18 and in at least two non-parallel linear axes (m, n). A stabilizing angle θ is shown in FIG. 12, which is defined by the non-parallel linear axes. FIG. 12 is provided for illustration purposes to explain the stabilizing angle. Embodiments with a stabilizing surface that contacts target surface at different points, which would also include a broad stabilizing surface laying on the target surface at numerous points, would still nonetheless meet the criteria of having at least two points that contact the target surface.

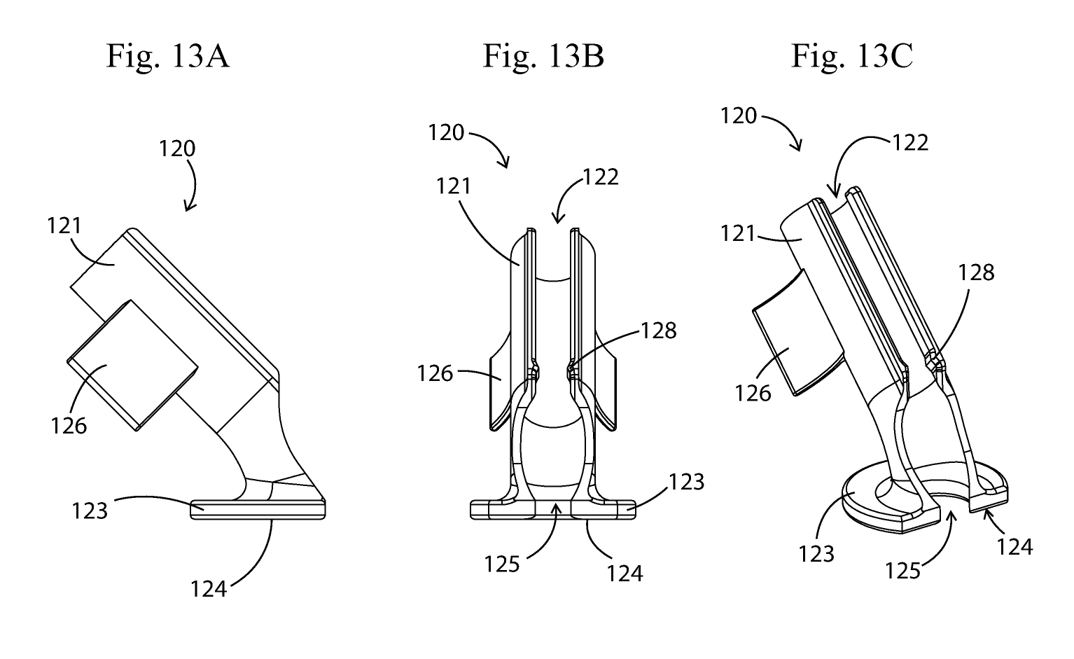

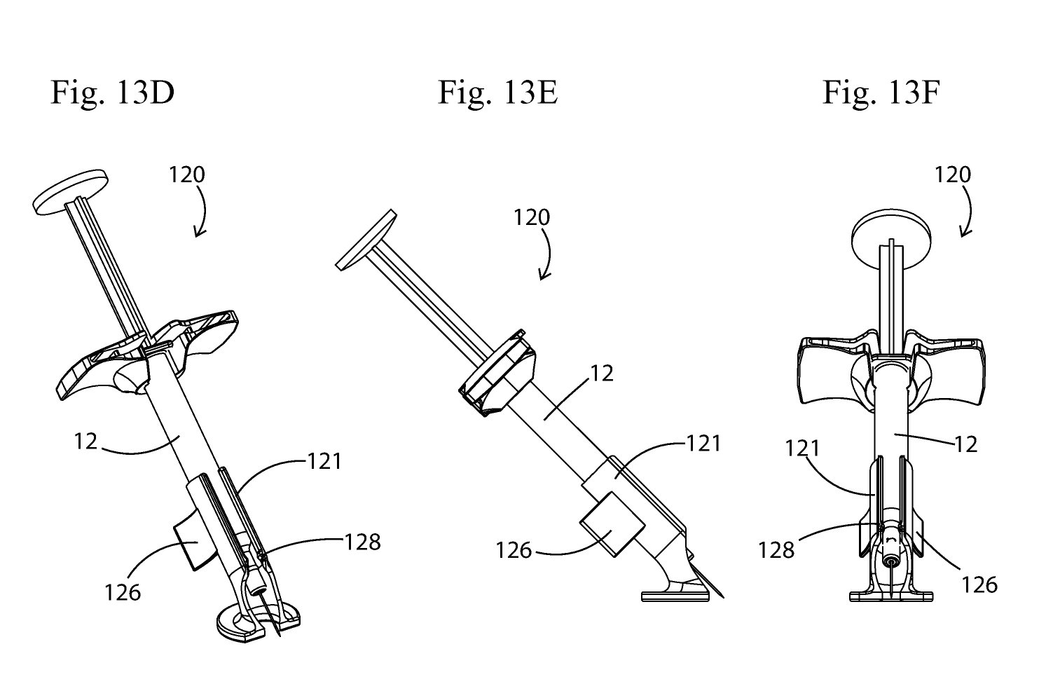

FIGS. 13A-13C provide side, front and perspective views of a further embodiment of a positioning apparatus 120 for directing or guiding an injection device to a target area for injection at a predetermined angle. The predetermined angle, in one non-limiting embodiment, may be formed between a stabilizing surface 124 and an angle of a channel 122 formed by a housing 121 of the positioning apparatus. The longitudinal axis of the channel 122 determines the angle at which the injection device 20 can be passed there through to the target surface of the user, in one non-limiting embodiment. In some non-limiting embodiments, this predetermined angle may be adjustable by a user. In other non-limiting embodiments, this angle may be fixed. The positioning apparatus 120 includes a housing 121 having a channel 122 terminating at an aperture 125 in a base 123 portion of the housing. The housing further includes a stabilizing surface 124. The positioning apparatus 120 further includes one or more tabs 126 for releasing the housing from an injection device 12 (not shown in FIGS. 13A-C) associated with the channel 122 of the housing 121.

During use of the positioning apparatus 120, an injection device 12, shown in FIGS. 13D-13F, is placed into the channel 122, such that the housing 121 becomes removably affixed to the injection device 12, the base 123 with stabilizing surface 124 is placed against the target surface of the user, such that the aperture 125 aligns with the target point on the target surface of the user. The injection device 12 is actuated, and following use of the injection device 12 via the positioning apparatus 120, the positioning apparatus 120 is removed from the injection device 12 by manipulation of one or more tabs 126 to release the housing 121 from the device 12. In one non-limiting embodiment, two tabs 126 are provided, and compression of the tabs 126 toward one another releases the housing 121 from the injection device 12. In one non-limiting embodiment, the tabs 126 are used to separate the apparatus 120 from the injection device 12 following use, in order to provide a hands-free drop of the injection device into a sharps container, for example. This particular safety feature prevents contact with an exposed needle, in order to minimize risk of exposure and needle pricks. In the embodiment of the positioning apparatus 120, one or more protrusions 128 are provided on the housing 121. The protrusions 128, shown in FIGS. 13B, 13C, 13D, and 13F project into the channel 122, such that an injection device 20 having, for example, a rigid needle shield on its distal end can be inserted into the channel 122, and the protrusions 128 can separate the rigid needle shield from the injection device as the injection device is inserted into the housing 121 prior to an injection with the injection device 20, in one non-limiting example. In one non-limiting embodiment, the protrusions 128 provide sufficient separation between the rigid needle shield and the remainder of the injection device 20 that a user a can easily remove the rigid needle shield from the injection device 20 before use, while the injection device 20 is within the housing 121. In another non-limiting embodiment, the protrusions 128 may serve to completely remove the rigid needle shield from the injection device 20 as the injection device 20 is inserted into the housing 121 as shown in FIGS. 13D-13F (rigid needle shield is not shown).

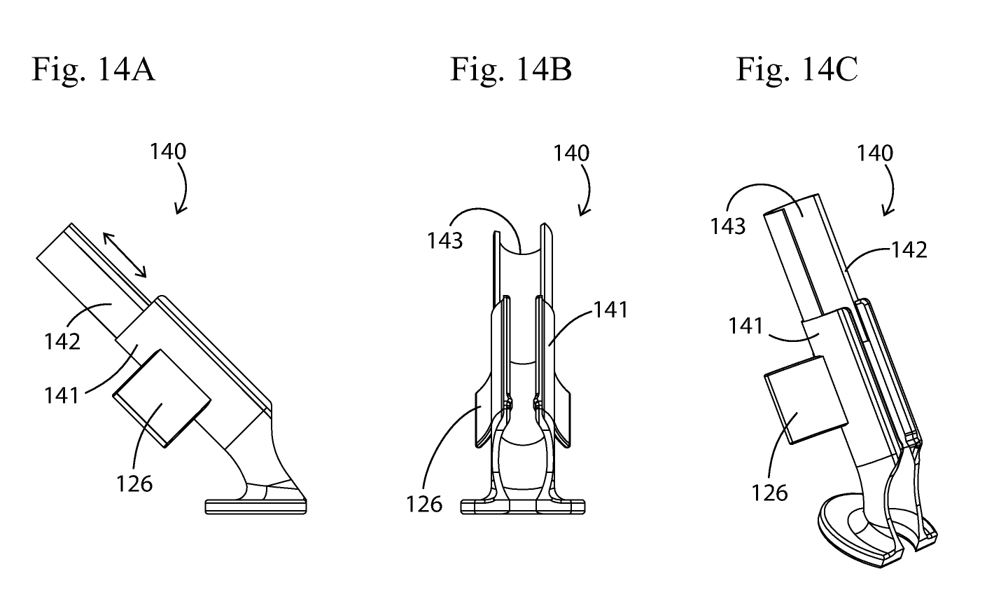

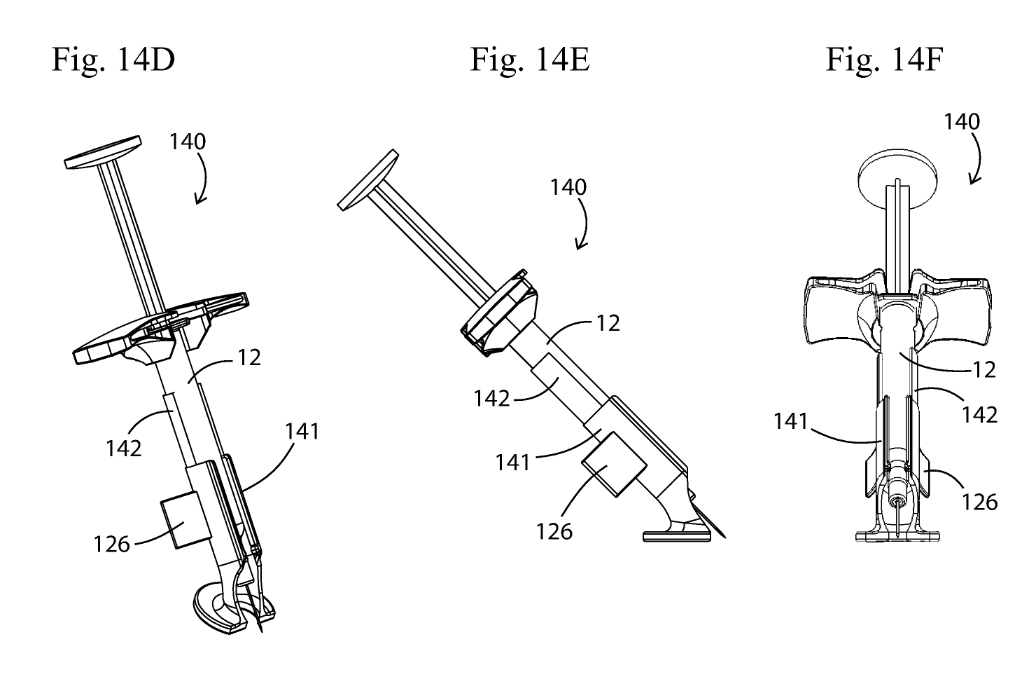

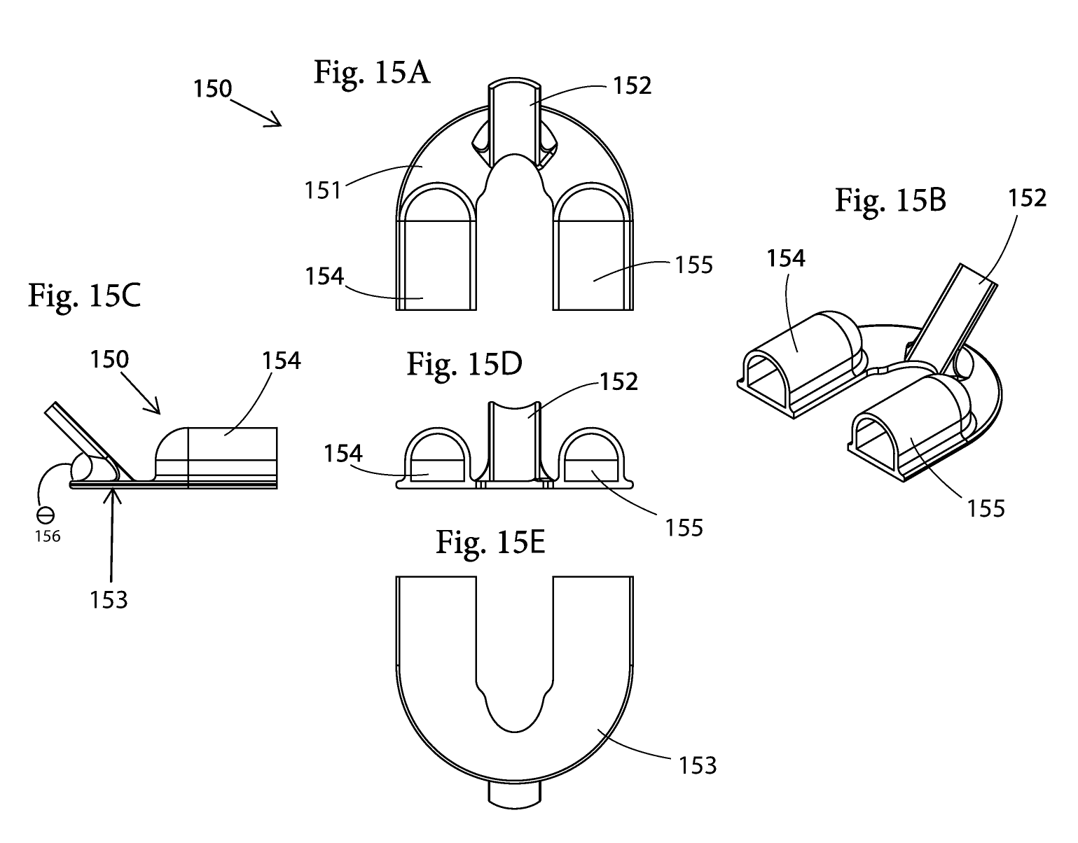

In a further embodiment shown in FIGS. 14A-14F, a positioning apparatus 140 is provided, wherein a guiding track 142 is associated with the housing 141 to guide an injection device 12 into the channel 143 of the apparatus 140. The guiding track 143 may also guide the injection device 12 along the longitudinal axis of the channel 143 and housing 141. The guiding track 142 may, in a non-limiting embodiment, move relative to the housing 141 to guide the injection device 12 toward the target surface of the user at a predetermined angle during use. In one particular non-limiting embodiment, the guiding track 142 may slide relative to the housing 141 in a first direction to deliver an injection device 12 to the target surface, and may slide in a second direction relative to the target surface to remove the injection device 12 from the target surface, for example.

In still a further embodiment, a positioning apparatus 150 for guiding an injection device relative to a target surface having a representative plane is provided. The apparatus 150 includes a housing 151 having a stabilizing surface 153, a guiding member 152 configured to receive and guide an injection device 12 (not shown) to a target surface at a first angle 156, wherein the housing 151 includes at least a first receiving member 154 and a second receiving member 155, the first and second receiving members 154, 155, being configured to surround a target area of the target surface during use. These receiving members 154, 155 may form a protective sleeve for the user to manipulate the positioning apparatus 150 at the target site for injection during use of the injection device. The receiving members 154, 155 may provide an added safety feature of protecting fingers of a user received within the receiving members 154, 155, in a non-limiting embodiment, from accidental needle prick by an injection device 12 (not shown) during use of the positioning apparatus with a needle-containing injection device, for example. In one non-limiting embodiment, at least a portion of the base portion of the housing 151 may include a flexible material, such that when a user inserts his or her fingers into the first and second receiving members 154, 155, and places the positioning apparatus 150 against the target surface, at least a portion of the target surface beneath the positioning apparatus 150 may be pinched between the first and second receiving members 154, 155, to target an injection at the target point when the injection device 12 is placed on the guiding member 152 and guided toward the target location.

In embodiments provided herein, a sensor 79 (as shown in FIG. 1A-1B, 2A-2B, 3A-3B) may be associated with the injection device 12 or the positioning apparatus, wherein the sensor 79 senses and records a use and/or a condition of the injection device 12 and/or of the positioning apparatus. One or more sensors may be incorporated into any of the embodiments described herein, not limited to the embodiments shown in FIGS. 1-3. In further embodiments, the sensor 79 may be configured to detect a condition or a use of the injection device 12 or the positioning apparatus based on a condition sensed by the sensor 79. In still a further embodiment, the sensor 79 may be configured to detect a condition of the housing and/or the injection device based on a condition detected by the sensor 79. In still a further embodiment, a condition may include an orientation, a position, a location, a temperature, a use, or a combination thereof, of the housing, positioning apparatus, and/or the injection device 12.

In further embodiments, the positioning apparatus may be associated with an information sending, detecting and/or receiving component configured to receive information or send information about the injection device and/or the housing related to the condition, and optionally further comprising at least one of: a signal output component, a microprocessor, a storage medium component, and a power source. In still a further embodiment, the positioning apparatus senses and records use of the positioning apparatus and/or the injection device. In yet a further embodiment, the signal output component is configured to output an audio, a visual, a tactile, a gustatory, or an olfactory signal, or a combination thereof, to a user.

In yet a further embodiment, the positioning apparatus may be configured to send information to and/or receive information from an external device via a wired or a wireless connection, wherein said connection provides a communication of power and/or information between the external device and the housing. The wireless connection may include a Bluetooth® and/or an RFID (Radio Frequency Identification) technology, in non-limiting embodiments. The RFID technology may include an RFID transponder and an RFID reader.

In another embodiment, the housing or the injection device further includes either a unique identification component, the unique identification component including information about a medicament, configured to be read by a unique identification reader associated with the injection device, or a unique identification reader configured to read information on a unique identification component associated with the injection device. In a further embodiment, the unique identification component may be a bar code and the unique identification reader is a bar code reader. In further embodiments, the positioning apparatus or injection device may include pre-programmed information about an injection device and/or a medicament. In still a further embodiment, the positioning apparatus, housing, or injection device may be configured to download or receive information from a database, the Internet, and/or another device, the information may include information about a medicament, information about an injection device including instructions for use, information about contraindication of medicaments, information about a user, medicament dosage information, storage information, prescribing physician information, pharmacy information, manufacturer information, warning information, recall information, environmental data, geographical information, time zone information, MET (meteorological) data, storage information, supply chain information, transit data, and/or temperature data.

In a further embodiment, the signal output component may include a speaker, a display, a light, a vibration component, a smell-emitting component, or a temperature-changing component, or a combination thereof. In still a further embodiment, the condition may include an error condition, a correct usage of the system, and/or an input sensed by the system. In y et a further embodiment, the positioning apparatus, an external device or the housing may provide a feedback to a user via a signal output component based on the condition detected. In still a further embodiment, the housing, positioning apparatus, injection device, or external device may provide instructions for using the injection device in a sequence of steps, and wherein the microprocessor is configured to control a provision of the instructions for using the injection device and/or the housing to the user in the sequence of steps and/or to provide an instruction to the user based on information detected and/or received by the information detecting and/or receiving component of the housing. In one non-limiting embodiment, the instructions may pertain to correct or incorrect angular positioning of the injection device relative to the target surface.

In a further embodiment, a visual indicator configured to provide correct angular alignment of the injection device relative to the target surface when the injection device is aligned with the visual indicator is provided. Alignment of the injection device with the housing or positioning apparatus as indicated by the visual indicator may provide correct alignment for proper angular alignment for an injection. In a further, non-limiting embodiment, the visual indicator may include a light and/or a marking on the housing and/or the injection device. It should be borne in mind that all patents, patent applications, patent publications, technical publications, scientific publications, and other references referenced herein are hereby incorporated by reference in this application in order to more fully describe the state of the art to which the present invention pertains.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise.

1. A positioning apparatus for guiding an injection device relative to a target surface having a representative plane, the apparatus comprising:
a housing defining a channel configured to receive an injection device, wherein a first angle is formed between a longitudinal axis of the channel and the representative plane of the target surface so as to guide the injection device through the channel to the target surface at the first angle, and wherein said housing comprises a stabilizing surface configured to abut the target surface to stabilize an injection according to said first angle.

2. The positioning apparatus of claim 1, wherein the first angle comprises about a 90 degree angle.

3. The positioning apparatus of claim 1, wherein the first angle comprises about a 45 degree angle.

4. The positioning apparatus of claim 1, wherein the first angle comprises between about a 10-15 degree angle.

5. The positioning apparatus of claim 1, wherein the first angle is less than 90 degrees.

6. The positioning apparatus of claim 1, wherein the first angle comprises an angle between 30 degrees and 90 degrees.

7. The positioning apparatus of claim 1, wherein the stabilizing surface reduces pivoting of the longitudinal axis of the channel relative to the target surface.

8. The positioning apparatus of claim 1, wherein the channel has a proximal end and a distal end, and wherein the stabilizing surface contacts the target surface at least two points, at least 1-5 cm from a center point of a distal opening of the channel and in at least two non-parallel linear axes.

9. The positioning apparatus of claim 8, wherein the linear axes form a stabilization angle there between that is at least 20 degrees.

10. The positioning apparatus of claim 1, wherein the stabilizing surface contacts the target surface at least three non-linear points, at least 1-5 cm from a center point of a distal opening of the channel.

11. The positioning apparatus of claim 1, wherein the first angle comprises an angle between about 40 degrees and about 50 degrees.

12. The positioning apparatus of claim 1, wherein the housing comprises one or more protrusions adjacent to the channel, said one or more protrusions are configured to retain the injection device within the housing when the injection device is received within the channel.

13. The positioning apparatus of claim 1, wherein the injection device comprises an injection training device.

14. The positioning apparatus of claim 1, wherein the injection device comprises a medicament delivery device, and wherein the channel is configured to guide the medicament delivery device to deliver an injection at a target point at the target surface, such that a longitudinal axis of the medicament delivery device is determined by the longitudinal axis of the channel, and the medicament delivery device contacts the target surface at the first angle.

15. The positioning apparatus of claim 14, further comprising an aperture formed in the stabilizing surface of the housing adjacent to the target surface, wherein when the injection device is received within the channel, a portion of a distal end of the injection device aligns with the aperture in the housing, such that an injection device comprising a needle at its distal end can exit the housing wherein the needle traverses the housing through the aperture.

16. The positioning apparatus of claim 15, wherein the injection device comprises a needle at its distal end, and wherein the needle is configured to traverse the housing through the aperture to a target point at the target surface.

17. The positioning apparatus of claim 14, further comprising a platform extending from the stabilizing surface, the platform comprising a longitudinal axis transverse to the longitudinal axis of the channel, wherein the platform is configured to abut the target surface.

18. The positioning apparatus of claim 17, wherein the platform comprises a first portion and a second portion, said first and second portions are disposed on either side of the housing, and said platform comprises a flexible material such that a user can grip the first and second portions of the platform to receive at least a portion of the target surface between the first and second portions of the platform to displace the portion of the target surface to receive the injection device.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. A method for providing control of an injection device at a target point of a target surface along a first angle relative to the target surface having a representative plane, said method comprising:
positioning a positioning apparatus comprising a housing having a guide and being configured to receive an injection device, wherein the guide forms a first angle between a longitudinal axis of the guide and the representative plane of the target surface so as to guide an injection device to the target surface at the first angle and wherein said housing comprises a stabilizing surface to stabilize an injection at the target surface according to said first angle, such that the stabilizing surface of the housing abuts the target surface, and a distal portion of the guide is in alignment with the target point of the target surface;
placing the injection device into the housing in alignment with the guide at the first angle; and
gliding the injection device to the target point.

53. The positioning apparatus of claim 1, wherein at least a portion of the stabilizing surface comprises an attachment component configured to removably affix at least the portion of the stabilizing surface to the target surface.

54. (canceled)

55. (canceled)

56. (canceled)

57. The positioning apparatus of claim 1, further comprising a sensor associated with the housing and/or the injection device, said sensor configured to detect a condition of the housing and/or the injection device based on a condition detected by the sensor.

58. (canceled)

59. The positioning apparatus of claim 57, wherein the positioning apparatus and/or the injection device is associated with an information sending, detecting and/or receiving component configured to receive information or send information about the injection device and/or the housing related to a condition of the injection device and/or the housing, and optionally further comprises at least one of: a signal output component, a microprocessor, a storage medium component, and a power source.

60. The positioning apparatus of claim 59, wherein the positioning apparatus senses and records a use of the positioning apparatus and/or the injection device.

61. The positioning apparatus of claim 59, said signal output component is configured to output an audio, a visual, a tactile, a gustatory, or an olfactory signal, or a combination thereof, to a user.

62. The positioning apparatus of claim 59, wherein the positioning apparatus is configured to send information to and/or receive information from an external device via a wired or a wireless connection, wherein said connection provides a communication of power and/or information between the external device and the positioning apparatus and/or the injection device.

63. (canceled)

64. (canceled)

65. The positioning apparatus of claim 1 wherein the positioning apparatus or the injection device further comprises either a unique identification component, said unique identification component comprising information about a medicament, configured to be read by a unique identification reader associated with the injection device, or a unique identification reader configured to read information on a unique identification component associated with the injection device.

66. (canceled)

67. The positioning apparatus of claim 1, further comprising pre-programmed information about an injection device and/or a medicament.

68. The positioning apparatus of claim 59, wherein the positioning apparatus and/or the injection device is configured to download or receive information from a database, the Internet, and/or another device, said information comprises information about a medicament, information about an injection device including instructions for use, information about contraindication of medicaments, information about a user, medicament dosage information, storage information, prescribing physician information, pharmacy information, manufacturer information, warning information, recall information, environmental data, geographical information, time zone information, MET (meteorological) data, storage information, supply chain information, transit data, and/or temperature data.

69. The positioning apparatus of claim 59, wherein the signal output component comprises a speaker, a display, a light, a vibration component, a smell-emitting component, or a temperature-changing component, or a combination thereof.

70. (canceled)

71. (canceled)

72. The positioning apparatus of claim 59, wherein the positioning apparatus and/or the injection device may provide instructions for using the positioning apparatus and/or the injection device, and wherein the microprocessor is configured to control a provision of the instructions for using the injection device and/or the positioning apparatus to a user and/or to provide an instruction to the user based on information detected and/or received by the information detecting and/or receiving component of the positioning apparatus and/or the injection device.

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. (canceled)

150. (canceled)

151. (canceled)

152. (canceled)

153. The positioning apparatus of claim 1, further comprising a guiding track associated with the housing, said guiding track being configured to receive at least a portion of the injection device, wherein the guiding track guides the injection device along the longitudinal axis of the channel.

154. The positioning apparatus of claim 153, wherein the guiding track is movable within the channel, relative to the housing, such that when the injection device is received within the guiding track, the guiding track is movable in a first direction toward the target surface and in a second direction away from the target surface.

155. (canceled)

156. (canceled)

157. (canceled)
